(19) United States
(12) Patent Application Publication
(10) Pub. No.: US 2025/0230139 A1
(43) Pub. Date: Jul. 17, 2025

(54) Novel Process and Intermediate for the Preparation of Apalutamide

(71) Applicant: BDR Lifesciences Private Limited, Padra, Vadodara (IN)

(72) Inventors: Dharmesh Mahendrabhai Shah, Mumbai (IN); Rajendrakumar Gokaldas Chavda, Padra, Vadodara (IN); Yashraj Anjankumar Majmudar, Padra, Vadodara (IN); Madhavkumar Dilipbhai Trivedi, Halol, Panchmahals (IN); Harshad Ghanshyambhai Kathrotiya, Padra, Vadodara (IN); Sagar Premjibhai Kothadiya, Padra, Vadodara (IN); Pratik Ashwinbhai Vora, Halol, Panchmahals (IN); Jaysukh Bhupatbhai Bhalala, Padra, Vadodara (IN)

(21) Appl. No.: 18/854,748

(22) PCT Filed: Apr. 8, 2023
(86) PCT No.: PCT/IN2023/050342

§ 371 Date: Oct. 7, 2024

(30) Foreign Application Priority Data
Apr. 8, 2022 (IN) 202221021077

(51) Int. Cl. C07D 401/04

(52) U.S. Cl. CPC C07D 401/04

(57) ABSTRACT

The present invention relates to novel highly efficient and economic process for large-scale production of Apalutamide. The present invention also relates to novel process that form highly pure Apalutamide through a novel intermediate. This process avoids use of expensive and hazardous reagents and solvents. Along with the ease of performance, the present invention process also gives high-purity final product with high yield. This makes the present invention highly cost-effective and time-efficient.

### FIELD OF THE INVENTION

The present invention relates to novel highly efficient, and economic process for large-scale industrial production of Apalutamide and pharmaceutically acceptable salt thereof. The present invention also relates novel process that produce highly pure Apalutamide through use of a novel intermediate.

### BACKGROUND OF THE INVENTION

Apalutamide is an Androgen Receptor (AR) inhibitor that binds directly to the ligand-binding domain of the AR. Apalutamide inhibits AR nuclear translocation, inhibits DNA binding, and impedes AR-mediated transcription.

A major metabolite, N-desmethyl apalutamide, is a less potent inhibitor of AR, and exhibited one-third the activity of Apalutamide in an in-vitro transcriptional reporter assay. Apalutamide administration caused decreased tumor cell proliferation and increased apoptosis leading to decreased tumor volume in mouse xenograft models of prostate cancer. Therefore, Apalutamide is approved for marketing in the treatment of patients with metastatic castration-sensitive prostate cancer and non-metastatic castration-resistant prostate cancer.

Apalutamide (CAS: 956104-40-8) is chemically known as 4-[7-[6-cyano-5-(trifluoromethyl)pyridin-3-yl]-8-oxo-6-thioxo-5,7-diazaspiro[3.4]octan-5-yl]-2-fluoro-N-methylbenzamide. It has chemical formula C₂₁H₁₅F₄N₅O₂S and molecular weight 477.44 g/mol. Apalutamide is structurally represented as below:

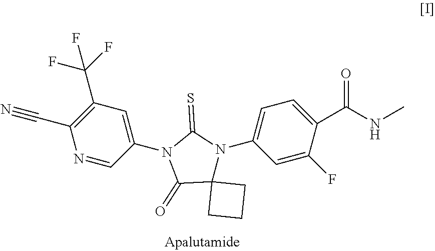 [SMILES: CNC(=O)c1ccc(N2C(=S)N(c3cnc(C#N)c(C(F)(F)F)c3)C(=O)C23CCC3)cc1F]

Apalutamide is non-hygroscopic white to slightly yellow powder. Apalutamide does not have a chiral center. Apalutamide melts at about 194-196° C. Apalutamide is practically insoluble in aqueous media over a wide range of pH values and practically insoluble to very soluble in organic solvents. Apalutamide has a dissociation constant pKa of 9.7 (acidic carboxamide moiety). The solubility experiments show that it is unstable to strong base (pH>11). Apalutamide exhibits polymorphism.

Apalutamide is the presently approved for marketing under brand name ERLEADA in USA since 14^{th} Feb., 2018 and in European Union since 14^{th} Jan., 2019. Apalutamide is provided as an immediate release, oblong shaped, greenish film-coated (FC) tablet for oral administration which contains 60 mg of Apalutamide drug substance. Inactive ingredients of this dosage form include colloidal anhydrous silica, croscarmellose sodium, hydroxypropyl methylcellulose-acetate succinate, magnesium stearate, microcrystalline cellulose, and silicified microcrystalline cellulose.

WO2006/124118 first time discloses lead compound for Apalutamide and process for preparation thereof.

Indian patent application no. 1920/DELNP/2015 (Corr. Patents: EP2368550, U.S. Pat. No. 8,445,507, WO2007/126765) discloses process for preparation of Apalutamide. This patent application discloses following process for preparation of Apalutamide as depicted in Scheme-1.

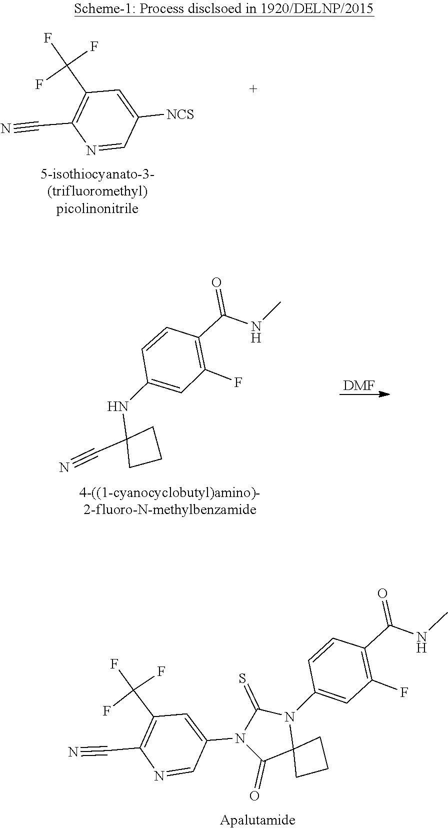

The process discloses the reaction of 5-isothiocyanato-3-(trifluoromethyl) picolinonitrile with 4-((1-cyanocyclobutyl)amino)-2-fluoro-N-methylbenzamide in dimethylformamide to obtain Apalutamide. The main disadvantage of this patent is that the use of dimethyl formamide in this reaction make the handling and work-up difficult. Hence, there is a need for the preparation of Apalutamide which is easy to handle.

WO2013/184681 discloses crystalline Forms viz. Form A, Form B, Form C, Form D, Form E, Form F, Form G, Form H, Form I and Form J of Apalutamide and process for preparation thereof. However, this prior-art failed to report stability study indicates less stable polymorphs at the long term storage.

WO2016/124149 discloses crystal Form I and Form II of Apalutamide and method of preparation thereof. Apalutamide obtained by the this patent method has a solid agglomeration and a non-uniform particle and has poor fluidity. Therefore, there is need for a robust process which produce Apalutamide and its polymorphs in a stable and pure form.

WO2018/136001 filed by Scinopharm Taiwan Ltd. discloses process for preparation of Apalutamide using condensation of following intermediates depicted in Scheme-2. This patent application is abandoned and hence, indicates inefficient development of Apalutamide process.

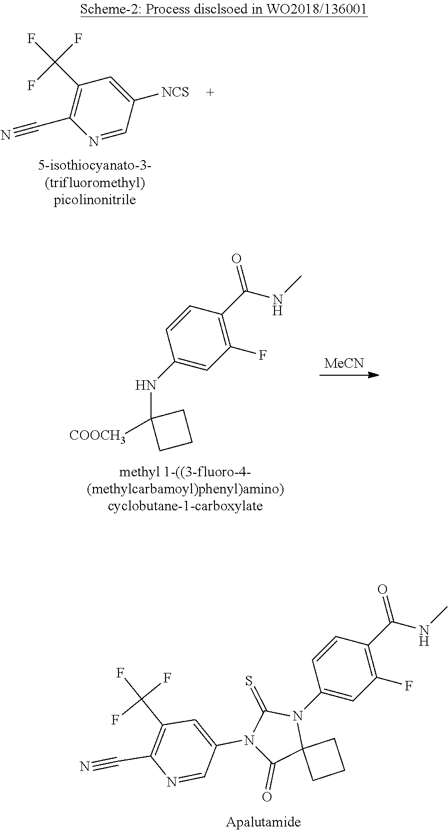

U.S. Ser. No. 10/807,965 filed by Cadila Healthcare Limited replaces methyl group to hydrogen in carbamoyl intermediate. This intermediate is condensed with isothiocyanate intermediate to product Apalutamide. The main disadvantage of this patent is that the said condensation process shall be done in the presence of wither phenol or alcohol, otherwise desired results are not found. The scheme-3 depicts the process for preparation of Apalutamide as described in this patent.

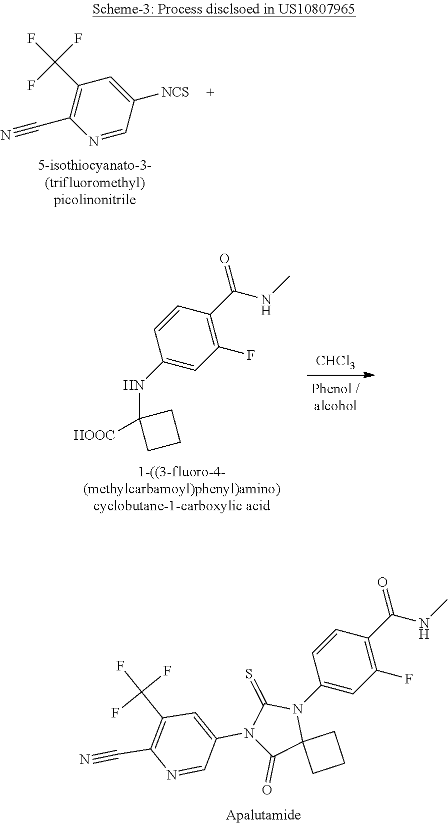

WO2019/229625 filed by Olon S.P.A. discloses the similar process in which intermediate having carboxyl activating group is condensed with isothiocyanate intermediate. This patent limits carboxyl activating group by three groups including —OMe, —SiOMe₃, —OC₆H₅. Scheme-4 depicts one aspect of this patent from three carboxyl activating groups. The main disadvantage of this this patent is that it has disclosed very low yiled ranging from 70-80% which is considered as very low at the industrial scale and hence, does not have indusctrial applicability.

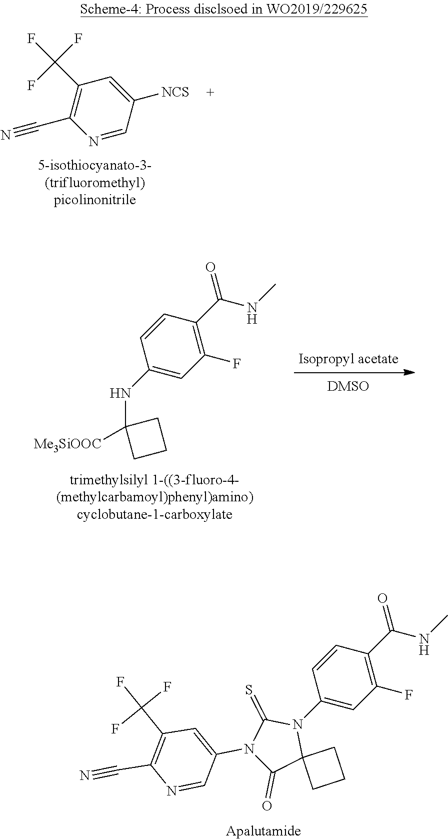

Indian Patent Application no. 202021006111 filed by Aarti Industries Limited discloses process for preparation of Apalutamide using novel intermedaite. However, limitation of the Indian patent application is that due to high stering hinderence in novel intermediate, strongest base like tertiary amine are used for the condensation process. The main disdvantage of this patent application is that without using expensive strong bases, this process may not work. Hence, there still an unmet need for novel process for the preparation of Apalutamide which is eco-friendly as well as economical and yet produce high purity compound with high yield. Scheme-5 depicts the process which is disclosed in Aarti Industries' patent application.

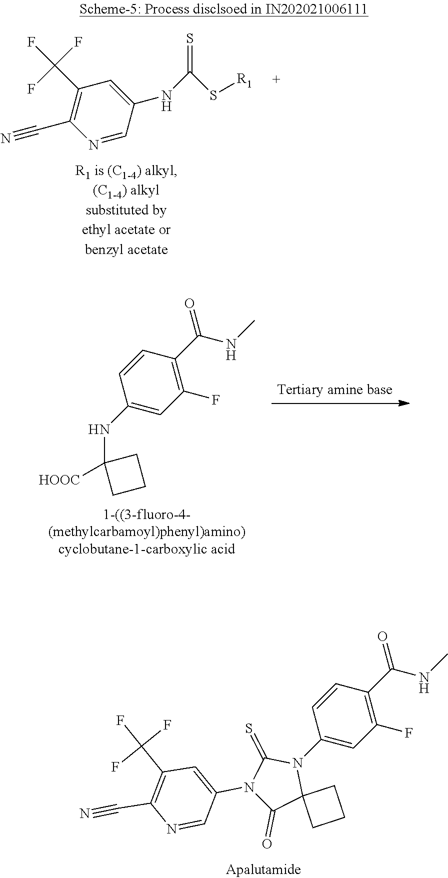

Therefore, as noted earlier although the above-mentioned various prior-arts already describe processes for manufacture of Apalutamide, although there is emerging need for new and improved process for manufacture of Apalutamide in high yield that is cost effective with high purity and less time consuming.

The inventors of the present invention have surprisingly developed novel process for manufacture of Apalutamide using a novel intermediate without use of expensive and hazardous reagent. This process uses very less number of quite cheap solvents and reagents that made the process highly cost-effective and eco-friendly. Along with the ease of performance, present invention process also gives high-purity final product with high yield.

Henceforth, a novel process in accordance with the present invention offers above remarkable advantages when compared to the processes already described in the prior-arts which makes the present process highly suitable for large scale industrial production.

### OBJECTIVE OF THE INVENTION

The principal objective of the present invention is to provide a process for preparation of Apalutamide to ameliorate any one of the disadvantages of the prior-art processes.

Yet another objective of the present invention is to provide an efficient, improved and industrially advantageous process for preparation of Apalutamide which is conveniently applicable to industrial scale.

Yet more objective of the present invention is to provide a process the preparation of Apalutamide having high purity and yield as well.

Another object of the present invention is to provide a novel process for the preparation of Apalutamide through a novel intermediate.

Another object of the present invention is to provide a cost-effective process for the preparation of Apalutamide which avoids the use of expensive reagent.

Another object of the present invention is to provide an eco-friendly process for the preparation of Apalutamide which uses very few numbers of solvent and reagents that are easily commercially available.

One more object of the present invention is to provide a process for the preparation of Apalutamide wherein each intermediate formed during the process are optionally isolated using various techniques in the prior art.

### SUMMARY OF THE INVENTION

The present invention discloses to novel, highly efficient and economic process for large-scale industrial production of Apalutamide. The present invention process is a cost-effective process that form highly pure Apalutamide through a novel intermediate with high yield.

One aspect of the present invention relates to preparation of Apalutamide comprising the following reaction:
a) 2-Fluoro-4-aminobenzoic acid ethyl ester (KSM-1) is condensed with bromocyclobutane-1-carboxylic acid (KSM-2) in presence of suitable base that leads to formation of a novel intermediate 1-[4-(ethoxycarbonyl)-3-fluoroanilino]cyclobutane-1-carboxylic acid (Intermediate-1);
b) 1-[4-(ethoxycarbonyl)-3-fluoroanilino]cyclobutane-1-carboxylic acid (Intermediate-1) is reacted with 5-isothiocyanato-3-(trifluoromethyl)pyridine-2-carbonitrile (KSM-3) in presence of suitable base that leads to formation of ethyl 4-(7-(6-cyano-5-(trifluoromethyl)pyridin-3-yl)-8-oxo-6-thioxo-5,7-diazaspiro[3.4]octan-5-yl)-2-fluorobenzoate (Intermediate-2);
c) ethyl 4-(7-(6-cyano-5-(trifluoromethyl)pyridin-3-yl)-8-oxo-6-thioxo-5,7-diazaspiro[3.4]octan-5-yl)-2-fluorobenzoate (Intermediate-2) is dissolved in suitable solvent in the presence of suitable base to form Apalutamide.

### DETAILED DESCRIPTION OF THE INVENTION

Accordingly, the present invention relates to a novel, economic and eco-friendly process for the preparation of Apalutamide that is highly suitable for large-scale industrial production of Apalutamide.

The present invention will now be disclosed by describing certain preferred and optional embodiments, to facilitate various aspects thereof.

References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

One embodiment of the present invention provides a process for the preparation of Apalutamide wherein Apalutamide is formed as highly pure form through a novel intermediate.

In one embodiment of the present invention, novel intermediate 1-[4-(ethoxycarbonyl)-3-fluoroanilino]cyclobutane-1-carboxylic acid is first time reported that is prepared by the condensation of 2-Fluoro-4-aminobenzoic acid ethyl ester (KSM-1) with bromocyclobutane-1-carboxylic acid (KSM-2) in appropriate solvents and reagents.

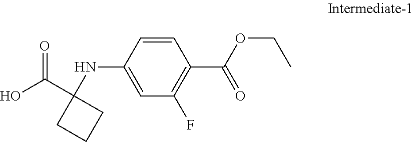 [SMILES: CCOC(=O)c1ccc(NC2(C(=O)O)CCC2)cc1F]

In another embodiment of the present invention, novel intermediate 1-[4-(ethoxycarbonyl)-3-fluoroanilino]cyclobutane-1-carboxylic acid is then reacted with 5-isothiocyanato-3-(trifluoromethyl)pyridine-2-carbonitrile (KSM-3) in suitable solvent and reagent to provide formation of ethyl 4-(7-(6-cyano-5-(trifluoromethyl)pyridin-3-yl)-8-oxo-6-thioxo-5,7-diazaspiro[3.4]octan-5-yl)-2-fluorobenzoate (Intermediate-2) through a novel process.

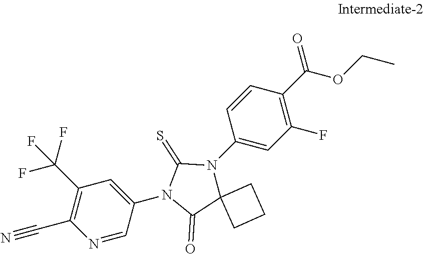 [SMILES: CCOC(=O)c1ccc(N2C(=S)N(c3cnc(C#N)c(C(F)(F)F)c3)C(=O)C23CCC3)cc1F]

In one more embodiment of the present invention, Apalutamide is formed through dissolution of ethyl 4-(7-(6-cyano-5-(trifluoromethyl)pyridin-3-yl)-8-oxo-6-thioxo-5,7-diazaspiro[3.4]octan-5-yl)-2-fluorobenzoate (Intermediate-2) in suitable solvents and reagents.

In one more embodiment of the present invention, final product formed using the process as per the present invention provides amorphous or crystalline Apalutamide using suitable solvents and reagents; preferably Crystalline Apalutamide Suitable solvents may include one or more of chloroform, tetrahydrofuran (THF), Dimethyl sulfoxide (DMSO), Dichloromethane (DCM), Ethyl acetate, n-heptane and combination thereof.

Suitable reagents or base may include one or more of methyl amine, triethylamine, di-isopropyl ethylamine and combination thereof.

The process of the present invention may be depicted as a whole in below scheme-6.

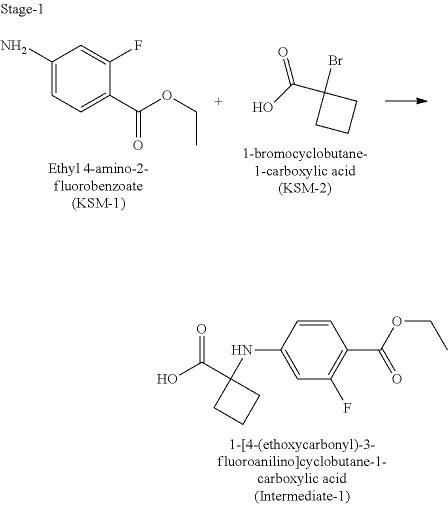 [SMILES: CCOC(=O)c1ccc(N)cc1F ; O=C(O)C1(Br)CCC1 ; CCOC(=O)c1ccc(NC2(C(=O)O)CCC2)cc1F]

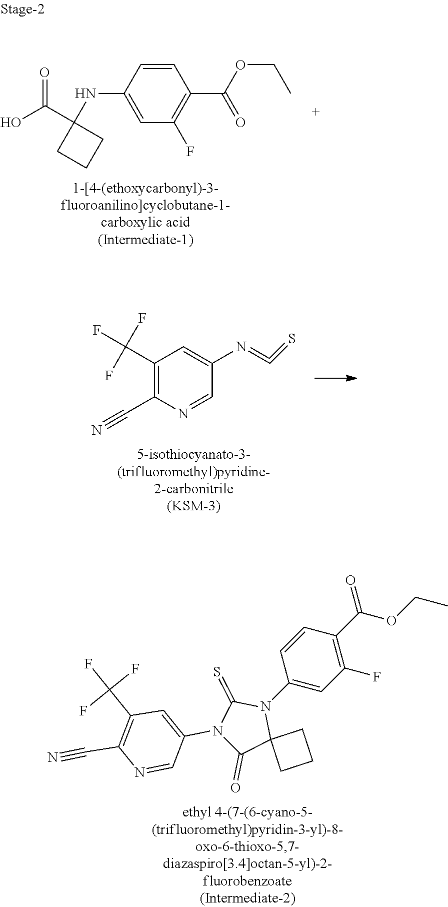 [SMILES: CCOC(=O)c1ccc(NC2(C(=O)O)CCC2)cc1F ; N#Cc1ncc(N=C=S)cc1C(F)(F)F ; CCOC(=O)c1ccc(N2C(=S)N(c3cnc(C#N)c(C(F)(F)F)c3)C(=O)C23CCC3)cc1F]

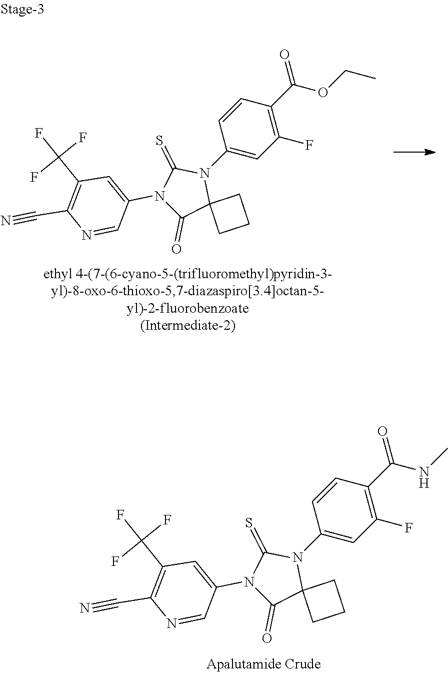 [SMILES: CCOC(=O)c1ccc(N2C(=S)N(c3cnc(C#N)c(C(F)(F)F)c3)C(=O)C23CCC3)cc1F ; CNC(=O)c1ccc(N2C(=S)N(c3cnc(C#N)c(C(F)(F)F)c3)C(=O)C23CCC3)cc1F]

In the present invention process, Apalutamide is prepared using novel process that form pure Apalutamide with high yield. This process is time-efficient as well as cost-effective as uses cheap and easily available solvents and reagents.

As per one embodiments of the present invention Apalutamide can be optionally purified to enhance purity and/or to remove impurity in the product. Any suitable purification method can be employed such as slurry wash, crystallization, base acid treatment and the like.

### EXAMPLES

Having described the invention with reference to certain preferred embodiments, other aspects will become apparent to one skilled in the art from consideration of the specification. The invention is further defined by reference to the following examples describing in detail by the preparation of the compounds of the invention. It will be apparent to those skilled in the art that many modifications, both to materials and methods, may be practiced without departing from the scope of the invention.

The following examples are provided for illustrative purpose only and these examples are in no way limitative on the present invention.

### Example-1

### Preparation of 1-[4-(ethoxycarbonyl)-3-fluoroanilino]cyclobutane-1-carboxylic acid (Intermediate-1)

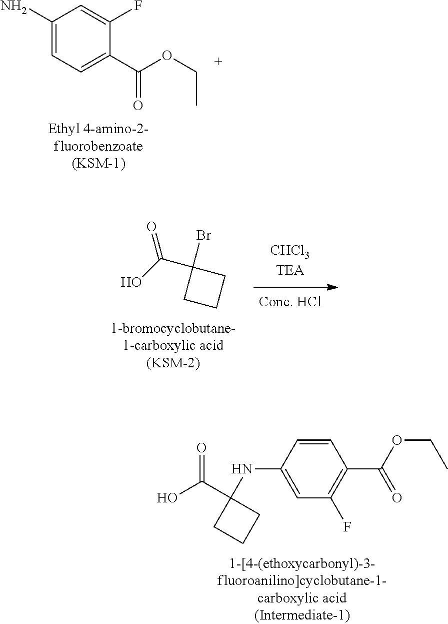 [SMILES: CCOC(=O)c1ccc(N)cc1F ; O=C(O)C1(Br)CCC1 ; CCOC(=O)c1ccc(NC2(C(=O)O)CCC2)cc1F]

100 gm of 2-Fluoro-4-aminobenzoic acid ethyl ester (KSM-1) and 117.3 gm of 1-bromocyclobutane-1-carboxylic acid (KSM-2) were condensed in the presence of 1000 ml chloroform and 132.25 ml triethylamine at 60-65° C. for 24 hrs. After completion of reaction added 100 ml concentrated HCl in to the reaction mass and distilled out chloroform under vacuum completely. Strip out the slurry reaction mass with 100 ml toluene. Add 300 ml toluene in to the reaction mass and heat to 65-70° C. Maintain reaction mixture for 30 min at 65-70° C. Cool the reaction mass to 25-30° C. and stir for 1 hr at 25-30° C. Filter the solid and wash with 100 ml toluene to get wet material. Dry the wet material at 50-55° C. in vacuum tray dryer to get 120 gm novel intermediate namely, 1-[4-(ethoxycarbonyl)-3-fluoroanilino]cyclobutane-1-carboxylic acid (Intermediate-1) (Purity 98.31%).

### Example-2

### Preparation of ethyl 4-(7-(6-cyano-5-(trifluoromethyl)pyridin-3-yl)-8-oxo-6-thioxo-5,7-diazaspiro[3.4]octan-5-yl)-2-fluorobenzoate (Intermedaite-2)

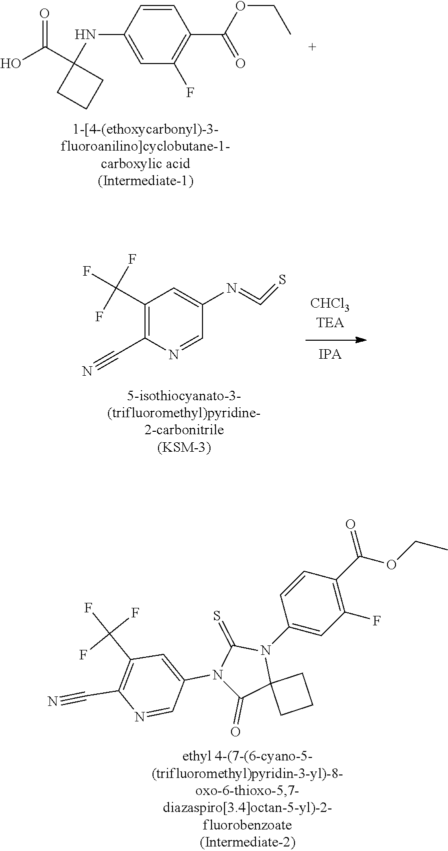 [SMILES: CCOC(=O)c1ccc(NC2(C(=O)O)CCC2)cc1F ; N#Cc1ncc(N=C=S)cc1C(F)(F)F ; CCOC(=O)c1ccc(N2C(=S)N(c3cnc(C#N)c(C(F)(F)F)c3)C(=O)C23CCC3)cc1F]

100 gm of 1-[4-(ethoxycarbonyl)-3-fluoroanilino]cyclobutane-1-carboxylic acid (Intermediate-1) and 97.76 gm of 5-isothiocyanato-3-(trifluoromethyl)pyridine-2-carbonitrile (KSM-3) were condensed in the presence of 1600 ml chloroform and 39.57 gm triethylamine at 60-65° C. After completion of reaction cool the reaction mass to 25-30° C. and filter it to remove salt. Take filtrate and distilled out chloroform under vacuum completely. Strip out the slurry mass with 100 ml IPA followed by addition of 400 ml IPA in to the reaction mass. Heat the reaction mass to 50-55° C. and stir for 30 min at 50-55° C. Cool the reaction mass to 25-30° C. and stir for 2 hrs at 25-30° C. Filter the solid and wash with 100 ml IPA to get wet material. Dry the wet material at 50-55° C. in vacuum tray dryer to produce 110 gm novel ethyl 4-(7-(6-cyano-5-(trifluoromethyl)pyridin-3-yl)-8-oxo-6-thioxo-5,7-diazaspiro[3.4]octan-5-yl)-2-fluorobenzoate (Intermediate-2) (Purity 97.2%).

### Example-3

### Preparation of Apalutamide

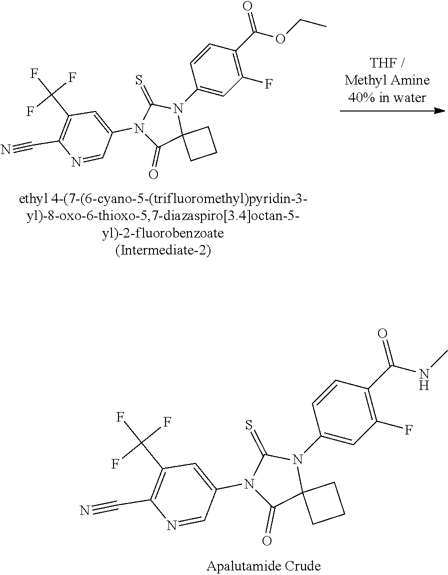 [SMILES: CCOC(=O)c1ccc(N2C(=S)N(c3cnc(C#N)c(C(F)(F)F)c3)C(=O)C23CCC3)cc1F ; CNC(=O)c1ccc(N2C(=S)N(c3cnc(C#N)c(C(F)(F)F)c3)C(=O)C23CCC3)cc1F]

100 gm of ethyl 4-(7-(6-cyano-5-(trifluoromethyl)pyridin-3-yl)-8-oxo-6-thioxo-5,7-diazaspiro[3.4]octan-5-yl)-2-fluorobenzoate (Intermediate-2) was stirred in presence of THF 200 ml tetrahydrofuran and 800 ml mono-methylamine at 5-10° C. for 1 hr. After completion of reaction, 500 ml water was added in to the reaction mass. Heat the reaction mass to 35° C. and distilled out solvent up to slurry mass at below 35° C. Cool the reaction mass to 15-20° C. and added HCl solution up to pH 8.0-10.0. Stir the reaction mass for 1 hr at 25-30° C. Filter the solid and wash with 100 ml IPA to get wet material. Dry the wet material at 50-55° C. in vacuum tray dryer to get 92.9 gm crude Apalutamide (Purity 90.72%).

### Example-4

### Preparation of Apalutamide

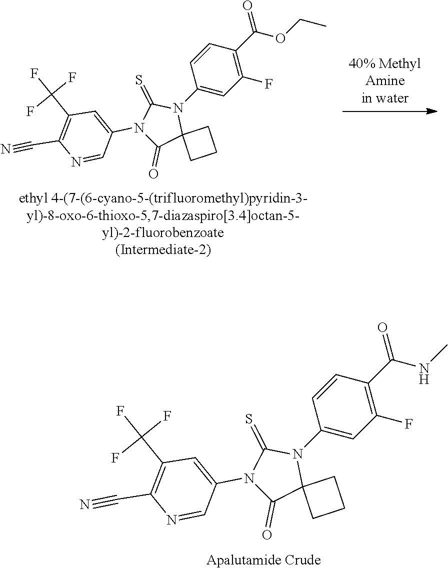 [SMILES: CCOC(=O)c1ccc(N2C(=S)N(c3cnc(C#N)c(C(F)(F)F)c3)C(=O)C23CCC3)cc1F ; CNC(=O)c1ccc(N2C(=S)N(c3cnc(C#N)c(C(F)(F)F)c3)C(=O)C23CCC3)cc1F]

100 gm of ethyl 4-(7-(6-cyano-5-(trifluoromethyl)pyridin-3-yl)-8-oxo-6-thioxo-5,7-diazaspiro[3.4]octan-5-yl)-2-fluorobenzoate (Intermediate-2) was stirred in 1500 ml mono-methylamine at 30-35° C. for 1 hr. After the completion of reaction, 500 ml water was added in to the reaction mixture. Distill out reaction mass to get slurry at below 35° C. Cool the reaction mass to 15-20° C. and added HCl solution up to pH 8.0-10.0. Stir the reaction mass for 1 hr at 25-30° C. Filter the solid and wash with 100 ml IPA to get wet material. Dry the wet material at 50-55° C. in vacuum tray dryer to get 95 gm crude Apalutamide (Purity 95.53%).

### Example-5

### Purification of Apalutamide

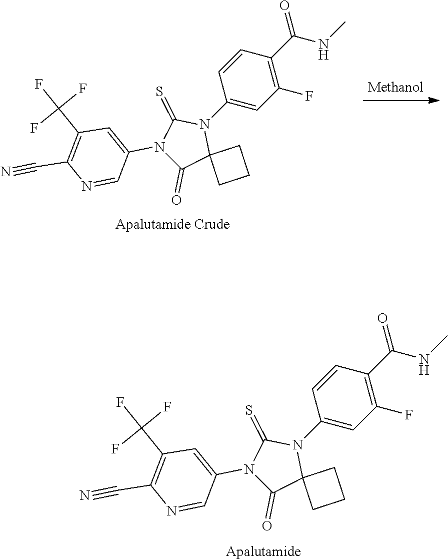 [SMILES: CNC(=O)c1ccc(N2C(=S)N(c3cnc(C#N)c(C(F)(F)F)c3)C(=O)C23CCC3)cc1F ; CNC(=O)c1ccc(N2C(=S)N(c3cnc(C#N)c(C(F)(F)F)c3)C(=O)C23CCC3)cc1F]

90 gm of Crude Apalutamide was added into 270 ml of Methanol and heat reaction mass at 60-65° C. to get clear reaction mass. 5% Activated charcoal treatment given at 60-65° C. Cool the reaction mixture to 20-25° C. followed by stir for 2 hrs at 20-25° C. Filter the solid and wash with 50 ml pre-chilled methanol to get wet material. Dry the wet material at 50-55° C. in vacuum tray dryer to get 72.5 gm gm pure Apalutamide (Purity 99.65%)

### Example-6

### Preparation of Apalutamide Form-B

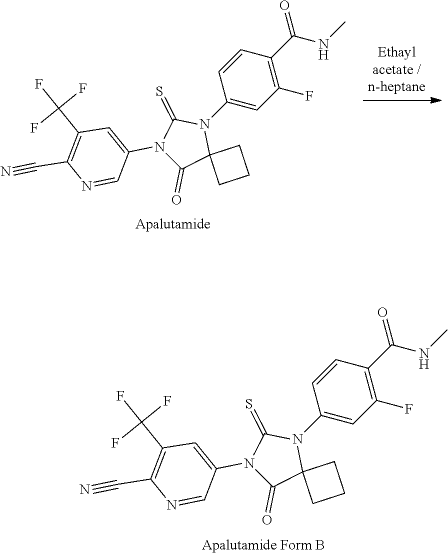 [SMILES: CNC(=O)c1ccc(N2C(=S)N(c3cnc(C#N)c(C(F)(F)F)c3)C(=O)C23CCC3)cc1F ; CNC(=O)c1ccc(N2C(=S)N(c3cnc(C#N)c(C(F)(F)F)c3)C(=O)C23CCC3)cc1F]

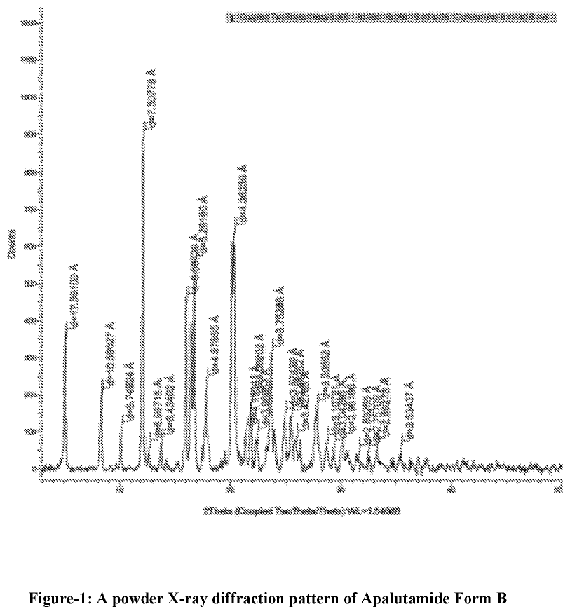

50 gm of Apalutamide (Obtained in ex. 5) was added into 150 ml Ethyl acetate and heat reaction mass at 60-65° C. to get clear reaction mass. 5% Activated charcoal treatment given at 60-65° C. Cool the reaction mixture to 25-30° C. followed by add 400 ml n-Heptane. Stir reaction mass for 2 hrs at 25-30° C. Filter the solid and wash with 50 ml n-Heptane to get wet material. Dry the wet material at 50-55° C. in vacuum tray dryer to get 45.3 gm pure Apalutamide Form-B (Purity 99.78%). FIG. 1 shows a powder X-ray diffraction pattern of Apalutamide Form B.

The invention described herein comprises in various objects and their description as mentioned above, with respect to characteristics and processes adopted. While these aspects are emphasised in the invention, any variations of the invention described above are not to be regarded as departure from the spirit and scope of the invention as described.

1. A novel process for preparation of apalutamide comprising of following steps:
a) reacting 2fluoro-4-aminobenzoic acid ethyl ester (KSM-1) in the presence of a first suitable solvent and a first suitable base with 1-bromocyclobutane-1-carboxylic acid (KSM-2) to obtain 1-[4-(ethoxycarbonyl)-3-fluoroanilino]cyclobutane-1-carboxylic acid (Intermediate-1); 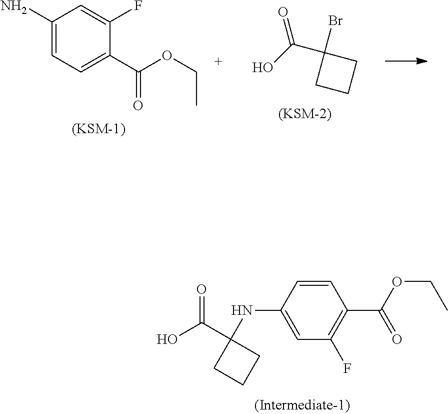
b) reacting 1-[4-(ethoxycarbonyl)-3-fluoroanilino]cyclobutane-1-carboxylic acid (Intermediate-1) in the presence of a second suitable solvent and a second suitable base with 5-isothiocyanato-3-(trifluoromethyl)pyridine-2-carbonitrile (KSM-3) to obtain ethyl 4-(7-(6-cyano-5-(trifluoromethyl)pyridin-3-yl)-8-oxo-6-thioxo-5,7-diazaspiro[3.4]octan-5-yl)-2-fluorobenzoate (Intermediate-2); 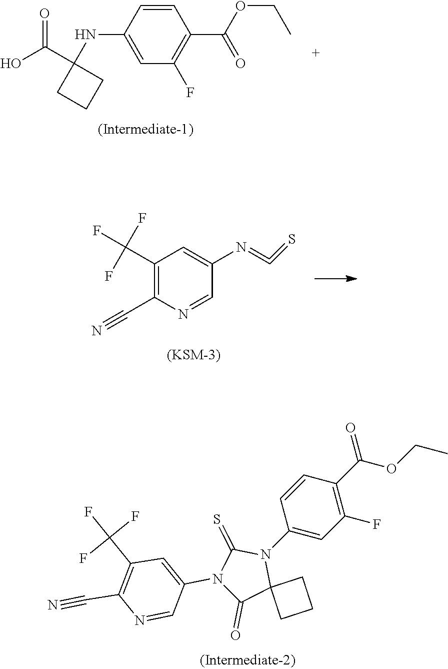
c) reacting ethyl 4-(7-(6-cyano-5-(trifluoromethyl)pyridin-3-yl)-8-oxo-6-thioxo-5,7diazaspiro[3.4]octan-5-yl)-2-fluorobenzoate (Intermediate-2) with mono-methylamine to obtain apalutamide;

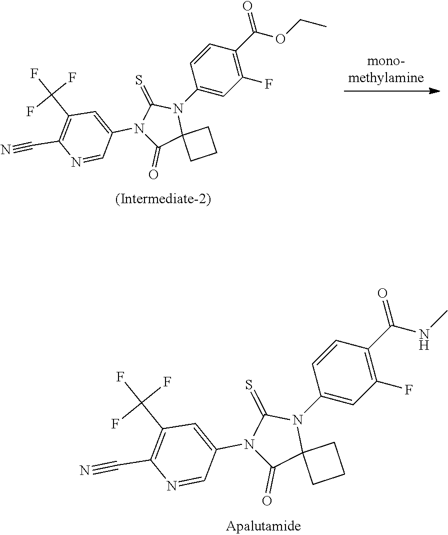 [SMILES: CCOC(=O)c1ccc(N2C(=S)N(c3cnc(C#N)c(C(F)(F)F)c3)C(=O)C23CCC3)cc1F ; CNC(=O)c1ccc(N2C(=S)N(c3cnc(C#N)c(C(F)(F)F)c3)C(=O)C23CCC3)cc1F]

2. The process as claimed in claim 1, wherein the first suitable solvent is selected from the group consisting of chloroform, tetrahydrofuran (THF), dimethyl sulfoxide (DMSO), dichloromethane (DCM), ethyl acetate, n-heptane, and a mixture thereof.

3. The process as claimed in claim 1, wherein the first suitable base is selected from methyl amine, triethylamine, di-isopropyl ethylamine, and a mixture thereof.

4. A novel compound 1-[4-(ethoxycarbonyl)-3-fluoroanilino]cyclobutane-1-carboxylic acid;

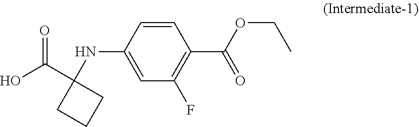 [SMILES: CCOC(=O)c1ccc(NC2(C(=O)O)CCC2)cc1F]

5. (canceled)

6. A process to prepare compound 1-[4-(ethoxycarbonyl)-3-fluoroanilino]cyclobutane-1-carboxylic acid (Intermediate-1) comprising reacting 2fluoro-4-aminobenzoic acid ethyl ester (KSM-1) in the presence of a suitable solvent and a suitable base with 1-bromocyclobutane-1-carboxylic acid (KSM-2);

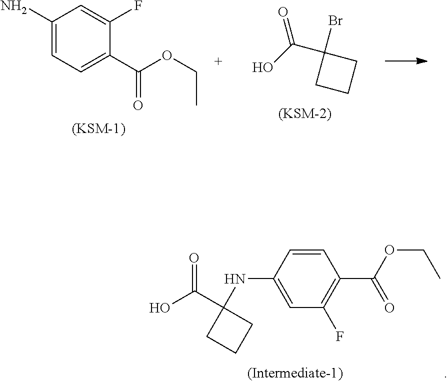

7. The process as claimed in claim 6, wherein the suitable solvent is selected from the group consisting of chloroform, tetrahydrofuran (THF), dimethyl sulfoxide (DMSO), dichloromethane (DCM), ethyl acetate, n-heptane, and a mixture thereof.

8. The process as claimed in claim 6, wherein the suitable base is selected from the group consisting of methyl amine, triethylamine, di-isopropyl ethylamine, and a mixture thereof.

9. The process as claimed in claim 6, wherein the suitable solvent is chloroform.

10. The process as claimed in claim 6, wherein the suitable base is triethylamine.

11. The process as claimed in claim 1, wherein the second suitable solvent is selected from the group consisting of chloroform, tetrahydrofuran (THF), dimethyl sulfoxide (DMSO), dichloromethane (DCM), ethyl acetate, n-heptane, and a mixture thereof.

12. The process as claimed in claim 1, wherein the second suitable base is selected from methyl amine, triethylamine, di-isopropyl ethylamine, and a mixture thereof.

13. The process as claimed in claim 1, wherein the first suitable solvent is chloroform.

14. The process as claimed in claim 1, wherein the first suitable base is triethylamine.

15. The process as claimed in claim 1, wherein the second suitable solvent is chloroform.

16. The process as claimed in claim 1, wherein the second suitable base is triethylamine.

17. The process as claimed in claim 1, wherein the first suitable solvent and the second suitable solvent are chloroform.

18. The process as claimed in claim 1, wherein the first suitable base and the second suitable base are triethylamine.
